(19) United States
(12) Patent Application Publication
(10) Pub. No.: US 2023/0334445 A1
(43) Pub. Date: Oct. 19, 2023

(54) METHOD OF MEDIATING PURCHASE OF PET MEDICATION AND SERVER THEREFOR

(71) Applicant: DD Cares Co.,Ltd, Seongnam-si (KR)

(72) Inventor: Sang Hyun KIM, Seongnam-si (KR)

(73) Assignee: DD Cares Co.,Ltd, Seongnam-si (KR)

(21) Appl. No.: 18/134,135

(22) Filed: Apr. 13, 2023

(30) Foreign Application Priority Data
Apr. 14, 2022 (KR) 10-2022-0046560

(51) Int. Cl. G06Q 20/10; G16H 20/10; G16H 40/67

(52) U.S. Cl. CPC G06Q 20/10; G16H 20/10; G16H 40/67

(57) ABSTRACT

Provided is a method of mediating purchase of a pet medication including: detecting an abnormality in the health status of a pet of a user, based on information collected by a wearable device; determining whether the duration of the detected abnormality exceeds a preset reference time period; determining a location of a user terminal by detecting an input of the user to the user terminal after the duration of the detected abnormality exceeds the reference time period; searching for location information of a plurality of veterinary pharmacies, based on the determined location, and transmitting, to the user terminal, a result of the searching; and receiving payment information input to the user terminal that has received the result, and transmitting the payment information to at least one terminal of the plurality of veterinary pharmacies.

### CROSS-REFERENCE TO RELATED APPLICATION

This application is based on and claims priority under 35 U.S.C. § 119 to Korean Patent Application No. 10-2022-0046560, filed on Apr. 14, 2022, in the Korean Intellectual Property Office, the disclosure of which is incorporated by reference herein in its entirety.

### BACKGROUND

### 1. Field

The present disclosure relates to a method of mediating between a user and a pharmacy for allowing the user to conveniently purchase a medication from the pharmacy through a user terminal, and more particularly, to a method of mediating between a user and a veterinary pharmacy for allowing the user to purchase a medication for pets through a user terminal, and a server for implementing the method.

### 2. Description of the Related Art

As of 2020, the number of households raising pets in Korea was estimated at over 6 million, which is more than 5 million as in 2019, and is converted into a population significantly greater than 10 million.

Accordingly, the pet product and pet service market for consumers raising pets is growing rapidly, and is expected to be worth more than 5 trillion won by 2025.

Regardless of the kind of pet, the lifespan of pets tends to be shorter than that of humans, and the environment for raising pets significantly varies depending on the kind or breed of pets, and thus, caregivers who put affection to pets while raising them tend to have a lot of interest in their companion pets' health status.

However, while caregivers raising pets are interested in the health status of their pets, there are only a limited number of pet health care services that respond to the caregivers' interest and thus, the need for a service for efficiently checking the health status of pets and maximizing the lifespan of pets is emerging.

### SUMMARY

Provided is a method of conveniently purchasing a veterinary medication through a terminal having a communication function.

Additional aspects will be set forth in part in the description which follows and, in part, will be apparent from the description, or may be learned by practice of the presented embodiments of the disclosure.

According to an aspect of an embodiment, a method of mediating purchase of a pet medication includes: detecting an abnormality in the health status of a pet of a user, based on information collected by a wearable device; determining whether the duration of the detected abnormality exceeds a preset reference time period; determining a location of a user terminal by detecting an input of the user to the user terminal after the duration of the detected abnormality exceeds the reference time period; searching for location information of a plurality of veterinary pharmacies, based on the determined location, and transmitting, to the user terminal, a result of the searching; and receiving payment information input to the user terminal that has received the result, and transmitting the payment information to at least one terminal of the plurality of veterinary pharmacies.

In the method, the payment information may be payment information for a veterinary medication corresponding to the abnormality.

In the method, the determining of the location of the user terminal may includes: when the duration exceeds the reference time period, activating a notification function of the user terminal; and determining the location of the user terminal by detecting the input of the user to the user terminal after the notification function is activated.

The method may further include transmitting, to the user terminal, location information of the veterinary pharmacy to which the payment information is transmitted.

In the method, the transmitting of the payment information may include transmitting the payment information to at least one terminal of a plurality of veterinary pharmacies that are selected in order of distance from the determined location.

In the method, the plurality of veterinary pharmacies may be in stock of the veterinary medication corresponding to the abnormality.

In the method, the plurality of veterinary pharmacies may deal in the veterinary medication corresponding to the abnormality.

In the method, the detecting of the abnormality may include receiving biosignals collected by the wearable device without passing through the user terminal.

In the method, the detecting of the abnormality may include receiving biosignals collected by the wearable device through the user terminal.

According to an aspect of another embodiment, a server for mediating purchase of a pet medication includes: an abnormality detection unit configured to detect an abnormality in the health status of a pet of a user, based on information collected by a wearable device; a reference determination unit configured to determine whether the duration of the detected abnormality exceeds a preset reference time period; a location determination unit configured to determine a location of a user terminal by detecting an input of the user to the user terminal after the duration of the detected abnormality exceeds the reference time period; a pharmacy location transmission unit configured to search for location information of a plurality of veterinary pharmacies, based on the determined location, and transmit, to the user terminal, a result of the searching; and a payment information transmission unit configured to receive payment information input to the user terminal that has received the result, and transmit the payment information to at least one terminal of the plurality of veterinary pharmacies.

In the server, the payment information may be payment information for a veterinary medication corresponding to the abnormality.

In the server, the location determination unit may be further configured to, when the duration exceeds the reference time period, activate a notification function of the user terminal, and determine the location of the user terminal by detecting the input of the user to the user terminal after the notification function is activated.

In the server, the payment information transmission unit may be further configured to transmit, to the user terminal, location information of the veterinary pharmacy to which the payment information is transmitted.

In the server, the payment information transmission unit may be further configured to transmit the payment information to at least one terminal of a plurality of veterinary pharmacies that are selected in order of distance from the determined location.

In the server, the plurality of veterinary pharmacies may be in stock of the veterinary medication corresponding to the abnormality.

In the server, the plurality of veterinary pharmacies may deal in the veterinary medication corresponding to the abnormality.

In the server, the abnormality detection unit may be further configured to receive biosignals collected by the wearable device without passing through the user terminal.

In the server, the abnormality detection unit may be further configured to receive biosignals collected by the wearable device through the user terminal.

According to an aspect of another embodiment, provided is a computer-readable recording medium having recorded thereon a program for executing the method.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram schematically illustrating an entire system for implementing a method according to the present disclosure;

FIG. 2 is a block diagram of modules constituting a management server according to the present disclosure;

FIG. 3 is a block diagram illustrating another example of a processing unit illustrated in FIG. 2;

FIG. 4 is a flowchart illustrating an example of a method according to the present disclosure; and

FIG. 5 is a flowchart illustrating another example of a method according to the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

As the present disclosure allows for various changes and numerous embodiments, particular embodiments will be illustrated in the drawings and described in detail. The effects and features of the present disclosure and methods of achieving them will become clear with reference to the embodiments described in detail below with the drawings. However, the present disclosure is not limited to the embodiments disclosed below, and may be implemented in various forms.

Hereinafter, embodiments of the present disclosure will be described in detail with reference to the accompanying drawings, and the same or corresponding components will be denoted by the same reference numerals when described with reference to the accompanying drawings, and thus, their descriptions that are already provided will be omitted.

In the following embodiments, terms such as “first,” “second,” etc., are used only to distinguish one component from another, and such components must not be limited by these terms.

In the following embodiments, the singular expression also includes the plural meaning as long as it is not inconsistent with the context.

In the following embodiments, the terms “comprises,” “includes,” “has”, and the like used herein specify the presence of stated features or components, but do not preclude the presence or addition of one or more other features or components.

When a certain embodiment may be differently implemented, particular operations may be performed differently from the sequence described herein. For example, two processes, which are successively described herein, may be substantially simultaneously performed, or may be performed in a process sequence opposite to a described process sequence.

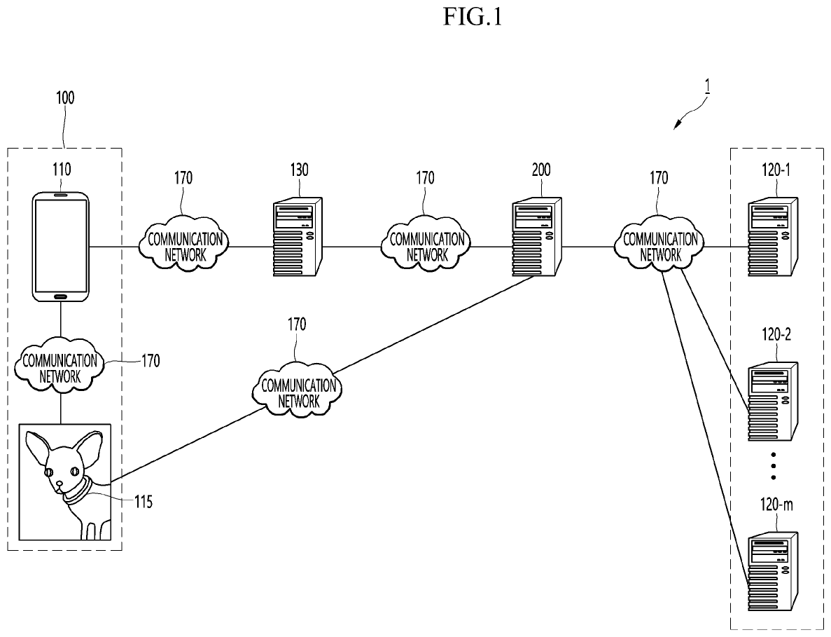

FIG. 1 is a diagram schematically illustrating an entire system for implementing a method according to the present disclosure.

It may be seen, from FIG. 1, that an entire system 1 according to an embodiment of the present disclosure has a structure in which a pet owner terminal group 100, a veterinary pharmacy terminal group 120, a communication provider server 130, and a management server 200 are connected to each other through a communication network 170.

First, the pet owner terminal group 100 may include an owner terminal 110 and a wearable device 115, and the owner terminal 110 and the wearable device 115 may be connected to each other through the communication network 170. The owner terminal 110 is a terminal used by an owner who owns a pet, and refers to an electronic device equipped with a communication module capable of communicating with the communication provider server 130 and the management server 200. Hereinafter, the owner refers to a person who owns a pet, and may be referred to as a user according to context.

The owner terminal 110 refers to a smart device including an input device for receiving an input of the owner, an output device (e.g., a display) for visually outputting an input to the owner terminal 110 or a processing result of the owner terminal 110, and a communication module capable of communicating with an external device, and thus, the size or type of the owner terminal 110 is not limited as long as it includes the input device, the output device, and the communication module. For example, although FIG. 1 illustrates the owner terminal 110 in the form of a smart phone, the owner terminal 110 may be a personal computer (PC), a notebook computer, a netbook, or the like capable of communicating with the communication provider server 130 and the management server 200.

The wearable device 115 refers to an electronic device that is wearable on the body of a pet to collect biosignals of the pet, and periodically or aperiodically transmit the collected biosignals to the owner terminal 110 or the management server 200. Hereinafter, the wearable device 115 will be abbreviated as the WD 115. The WD 115 may communicate with the owner terminal 110 through short-range communication such as Bluetooth, Wi-Fi, or near-field communication (NFC), and may communicate with the management server 200 through the communication network 170 rather than through the owner terminal 110.

Although FIG. 1 illustrates that the WD 115 is implemented as a collar attached to a dog's neck, but the WD 115 is not limited thereto and may be implemented to be attached to various parts such as a dog's ankle or torso. In addition, although FIG. 1 illustrates that the WD 115 has only one body, the WD 115 is not limited thereto and may be a device having a plurality of parts according to embodiments. In addition, although FIG. 1 illustrates that the pet wearing the WD 115 is a dog, but the pet in the present disclosure is not limited thereto and may be a cat, an iguana, a goldfish, a lizard, a hamster, or the like according to an embodiment.

The veterinary pharmacy terminal group 120 refers to a group of terminals of at least one veterinary pharmacy. Referring to FIG. 1, the veterinary pharmacy terminal group 120 includes m terminals, but is not limited thereto. Referring to FIG. 1, the veterinary pharmacy terminal group 120 includes terminals operated by different entities, such as a terminal 120-1 operated by a first veterinary pharmacy and a terminal 120-2 operated by a second veterinary pharmacy. An entity operating a veterinary pharmacy may apply a particular input to a terminal of the veterinary pharmacy capable communicating with the management server 200, to sell, to the owner, a veterinary medication that is in its stock or that is not in its inventory but may be stocked as the veterinary pharmacy deals in the veterinary medication. The terminal of each veterinary pharmacy included in the veterinary pharmacy terminal group 120 is illustrated as a server in FIG. 1, but is not limited thereto. In other words, the terminal of each veterinary pharmacy included in the veterinary pharmacy terminal group 120 is illustrated as a server, but the terminal of the veterinary pharmacy may be a PC, a notebook computer, a netbook or the like capable of communicating with the management server 200.

In the present disclosure, the management server 200 may select one of the veterinary pharmacies included in the veterinary pharmacy terminal group 120 and provide information about the selected veterinary pharmacy to the owner terminal 110 to allow the owner to visit the veterinary pharmacy and obtain the veterinary medication.

As an alternative embodiment, although not illustrated in FIG. 1, the veterinary pharmacy terminal group 120 may further include a veterinary medication stock management system (not shown). In the alternative embodiment, the management server 200 and the veterinary medication stock management system (not shown) operate in association with each other, and when the management server 200 requests a list of veterinary pharmacies where a veterinary medication selected by the owner is available, the veterinary medication stock management system (not shown) may read a command input to the management server 200 through an application programming interface (API) to search for list information about veterinary pharmacies across the country managed by the veterinary medication stock management system (not shown), and transmit a result of the searching to the management server 200.

The communication provider server 130 refers to a server operated by a communication provider communicating with the owner terminal 110. Here, the communication provider may be an internet service provider (ISP) capable of activating communication functions of the owner terminal 110 and the WD 115 according to a rate plan that the owner paid for. The owner may visit a nearby communication provider agency or call a customer service center operated by a communication provider, sign a certain communication contract, and set the communication function of the owner terminal 110 to be activated, and may also sign a communication contract for the WD 115 as an additional contract.

The owner may obtain the WD 115 equipped with a module (e.g., a universal subscriber identity module (USIM)) capable of communicating with the communication provider server 130 while signing the communication contract at the communication provider agency. After signing the communication contract, the WD 115 is able to communicate with the management server 200 through the communication network 170 operated by the communication provider server 130, and biosignals of the pet collected by the WD 115 may be periodically or aperiodically transmitted to the management server 200 in a state in which security is maintained. The WD 115 may independently communicate with the management server 200 through the communication network 170 operated by the communication provider server 130 without passing through the owner terminal 110, by generating and transmitting a signal including an identification code identifiable by the communication provider server 130.

According to the present disclosure, the owner may visit a nearby communication provider agency, sign a communication contract for the WD 115, and receive the WD 115 capable of communicating with the management server 200 through the communication network 170 provided by the communication provider server 130, and may use a supplementary service for purchasing a pet medication according to the present disclosure by simply visiting any one of communication provider agencies distributed throughout the country. In a case in which the WD 115 is a dedicated device provided by the communication provider agency, reliability may be guaranteed, and it is convenient for the owner to, at the communication provider agency, sign up for an insurance product that pays insurance money when the conditions are met based on the collected biosignals of the pet. In addition, according to an embodiment, the WD 115 may be a general communication device that is sold at an electronics store and may be equipped with a USIM.

The management server 200 may perform functions of detecting an abnormality in the health status of the owner's pet based on the biosignals received from the WD 115, and performing mediation to allow the owner to purchase a veterinary medication from a veterinary pharmacy. In detail, the management server 200 may periodically or aperiodically collect biosignals for estimating the health status of a pet, by receiving the biosignals of the pet directly from the WD 115 or receiving, through the owner terminal 110, the biosignals collected by the WD 115, and when an abnormality in the health status of the pet occurs, provide convenience to the owner to easily purchase a veterinary medication from a nearby veterinary pharmacy, and may indirectly contribute to an increase in sales of veterinary pharmacies. Detailed processes for implementing the above operation will be described below.

The communication network 170 performs a function of connecting the owner terminal 110, the WD 115, the communication provider server, the communication provider server 130, and the management server 200, which are components of the entire system 1 illustrated in FIG. 1, and may include various wired and wireless communication networks such as a data network, a mobile communication network, or an Internet network. In particular, in the present disclosure, the communication network 170 includes not only mobile communication networks currently in use, but also old-generation deprecated mobile communication networks, and thus may be any one of a Global System for Mobile communications (GSM) network, a code-division multiple access (CDMA) network, a wideband CDMA (WCDMA) network, a CDMA2000 network, a Long-Term Evolution (LTE) network, an LTE-Advanced (LTE-A) network, a 5th Generation (5G) network, and a 6th Generation (6G) mobile communication network expected to be serviced in 2030.

A summary of the processes of the entire system 1 illustrated in FIG. 1 is as follows.

Biosignals may be collected by the WD 115 worn by a pet and transmitted to the management server 200, and the management server 200 may analyze data regarding the biosignals to detect that an abnormality has occurred in the health of the pet. The management server 200 provides a notification to the owner terminal 110, determines the location of the owner terminal 110, searches for a plurality of veterinary pharmacies near the owner terminal 110, and provides a result of the searching to the owner terminal 110. The owner pays in advance for a veterinary medication available at at least one of the plurality of veterinary pharmacies displayed on the owner terminal 110, and visits the veterinary pharmacy to obtain the veterinary medication.

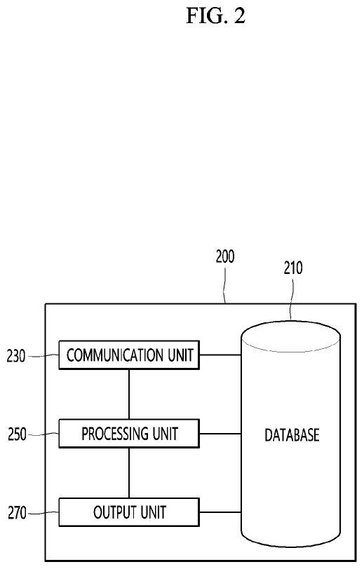

FIG. 2 is a block diagram of modules constituting a management server according to the present disclosure.

It may be seen, from FIG. 2, that the management server 200 includes a database 210, a communication unit 230, a processing unit 250, and an output unit 270. Hereinafter, descriptions will be provided with reference to FIG. 1.

The management server 200 according to an embodiment of the present disclosure may correspond to one or more processors, or may include one or more processors. Accordingly, the management server 200 and the communication unit 230, the processing unit 250, and the output unit 270 included in the management server 200 may be driven in a form included in a hardware device such as a microprocessor or a general-purpose computer system.

Each module included in the management server 200 illustrated in FIG. 2 is arbitrarily named in order to intuitively describe the representative function performed by the module, and in an actual implementation of the management server 200, each module may be named differently from as illustrated in FIG. 2.

In addition, the number of modules included in the management server 200 of FIG. 2 may vary according to an embodiment. In more detail, although FIG. 2 illustrates that the management server 200 includes a total of four modules, according to an embodiment, two or more modules may be integrated into one module, or one or more modules may be separated into two or more modules.

The database 210 stores various types of data necessary for the management server 200 to operate. For example, the database 210 stores an integrated management program for controlling the operation of the management server 200, and the database 210 may receive and store data received by the communication unit 230.

The communication unit 230 performs communication with the owner terminal 110, the WD 115, the veterinary pharmacy terminal group 120, and the communication provider server 130.

The processing unit 250 processes data received by the communication unit 230 and data to be transmitted. For example, the processing unit 250 may analyze and process biosignal data of the WD 115, which is received by the communication unit 230, to detect whether an abnormality has occurred in the health status of the pet. As another example, the processing unit 250 may determine the location of the owner terminal 110 based on the information (e.g., global positioning system (GPS) information) received from the owner terminal 110 having received the notification, and search for veterinary pharmacies near the owner terminal 110. In the above-described embodiments, the processing unit 250 performs a function of controlling overall data processing necessary to implement the method according to the present disclosure.

The output unit 270 performs functions of receiving a command from the processing unit 250 and generating and outputting various types of data. For example, the output unit 270 may output result data processed by the processing unit 250 and transmit the result data to the communication unit 230.

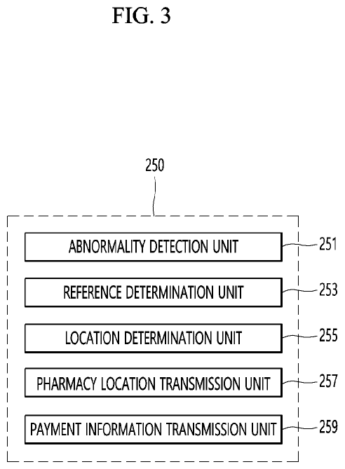

FIG. 3 is a block diagram illustrating another example of the processing unit described above with reference to FIG. 2.

It may be seen, from FIG. 3, that the processing unit 250 includes an abnormality detection unit 251, a reference determination unit 253, a location determination unit 255, a pharmacy location transmission unit 257, and a payment information transmission unit 259. The abnormality detection unit 251, the reference determination unit 253, the location determination unit 255, the pharmacy location transmission unit 257, and the payment information transmission unit 259 are sub-modules included in the processing unit 250, and thus may correspond to one or more processors as described above, or may be modules each including one or more processors and thus capable of performing independent operations. In addition, although FIG. 3 illustrates that the processing unit 250 includes a total of five sub-modules, according to an embodiment, one or more of the modules illustrated in FIG. 3 may be included in another module, or two or more of the modules illustrated in FIG. 3 may be integrated into one module. Hereinafter, descriptions will be provided with reference to FIGS. 1 and 2.

The abnormality detection unit 251 performs a function of detecting an abnormality in the health status of the owner's pet by using information collected by the WD 115. The abnormality detection unit 251 may receive information received by the communication unit 230 from the WD 115, identify information about biosignals of the pet from the received information, and detect whether an abnormality has occurred in the health of the pet, based on the identified information about the biosignals. The appearance of the WD 115 and the types of sensors included therein vary depending on the kind and breed of the pet, and the abnormality detection unit 251 includes an algorithm and reference data for identifying the owner and the pet based on identification information such as the serial number of the WD 115, and analyzing biosignals of the pet based on analysis criteria that vary depending on the owner and the pet.

The reference determination unit 253 performs functions of, when an abnormality is detected in the health status of the pet, additionally measuring the duration of the abnormality and determining whether the measured duration exceeds a preset reference time period. The health status of the pet may temporarily go into an abnormal state, but the owner may improve the health status of the pet based on pet-raising knowledge. Therefore, the reference determination unit 253 may detect that the health status of the pet has deteriorated and remains for a preset time period or longer, rather than temporarily deteriorating, and estimate that a veterinary medication is needed.

When the duration of the abnormality in the health status of the pet exceeds the reference time period, the location determination unit 255 may detect an input of the user to the owner terminal 110 after the duration exceeds the reference time period, and determine the location of the user terminal. For example, when the duration of the abnormality exceeds the reference time period, the location determination unit 255 may generate a notification signal and transmit the notification signal to the owner terminal 110 through the communication unit 230, to induce the owner terminal 110 to provide the owner with a visual, auditory, or tactile notification. The owner may check the notification output from the owner terminal 110 and release a standby mode (e.g., a sleep mode) of the owner terminal 110 by, for example, releasing a lock screen of the owner terminal 110, and in this process, an application (e.g., an app) installed in the owner terminal 110 may perform control such that information (e.g., coordinates, GPS values, longitude and latitude, etc.) for determining the location of the owner terminal 110 is transmitted to the location determination unit 255.

The pharmacy location transmission unit 257 may perform functions of searching for location information of a plurality of veterinary pharmacies based on the location of the owner terminal 110 determined by the location determination unit 255, and controlling the communication unit 230 to transmit a result of the searching to the owner terminal 110. The pharmacy location transmission unit 257 may search for a veterinary pharmacy located close to the owner terminal 110 within a preset distance, based on information about veterinary pharmacies stored in the database 210 or received from an external device (not shown), transmit a result of the searching to the owner terminal 110. The owner having first received the notification may be aware that the abnormality has occurred in the health status of the pet, then informed of the location of the veterinary pharmacy, and thus quickly visit the veterinary pharmacy to purchase a veterinary medication.

The payment information transmission unit 259 may receive payment information input to the owner terminal 110 and transmit the payment information to at least one terminal of a plurality of veterinary pharmacies. The owner may search for, through the owner terminal 110, a veterinary medication that may improve the health status of the pet, visit a veterinary pharmacy where the veterinary medication is available, and obtain the veterinary medication. In this case, the owner may obtain the veterinary medication by prepaying the price of the veterinary medication before visiting the veterinary pharmacy and then receiving the veterinary medication immediately after visiting the veterinary pharmacy. The above method of obtaining the veterinary medication may greatly improve the owner's convenience in that it may fundamentally prevent a case in which the owner visits the veterinary pharmacy but cannot purchase the veterinary medication because the veterinary pharmacy is out of stock. In addition, according to the method according to the present disclosure, an effect of increasing sales of veterinary pharmacies included in the veterinary pharmacy terminal group 120 may be expected.

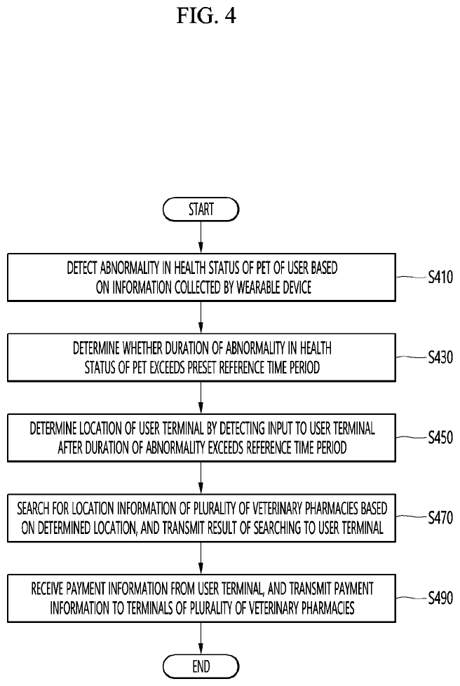

FIG. 4 is a flowchart illustrating an example of a method according to the present disclosure.

The method of FIG. 4 may be performed by the processing unit 250 described above with reference to FIGS. 1 to 3 and sub-modules included in the processing unit 250, and thus, hereinafter, descriptions will be provided with reference to FIGS. 1 to 3, and the descriptions provided above with reference to FIGS. 1 to 3 will be omitted.

The abnormality detection unit 251 may detect an abnormality in the health status of a pet of a user based on information collected by the WD 115 (S410). The abnormality detection unit 251 stores various reference values for the kind and breed of the pet in order to accurately determine an abnormality in the health status of the pet, and when biosignal data is collected by the WD 115, may identify the kind, breed, age, and other characteristics of the pet to detect (or determine) whether there is an abnormality in the health status of the pet.

The reference determination unit 253 may determine whether the duration of the abnormality in the health status of the pet exceeds a preset reference time period (S430). Here, the preset time period is a preset value to be compared with the duration for which the abnormality in the health status of the pet is maintained, and may be a numerical value stored in at least one of the database 210 and the reference determination unit 253. In the present disclosure, the module in which the reference time period is stored is not limited to a particular module, and thus, the reference time period may be stored in a module other than the above-described database 210 and reference determination unit 253.

The location determination unit 255 may determine the location of the user terminal (i.e., the owner terminal) by detecting an input to the user terminal after the duration of the abnormality in the health status of the pet exceeds the reference time period (S450).

For example, the location determination unit 255 may activate a notification function of the user terminal when the duration exceeds the reference time period, detect an input of the user to the user terminal after the notification function is activated, and thus determine the location of the user terminal. When a notification signal generated by the location determination unit 255 is transmitted to the user terminal through the communication unit 230, a visual, auditory, or tactile notification that may be confirmed by the user may be displayed on the user terminal. When the user makes an input to accept collection of location information of the user terminal, or the user actively inputs, to the user terminal, the location of the user terminal, the location determination unit 255 may detect the input to the user terminal, receive information corresponding to the input, and thus determine the location of the user terminal.

The pharmacy location transmission unit 257 searches for location information of a plurality of veterinary pharmacies based on the location of the user terminal determined by the location determination unit 255, and transmits a result of the searching to the user terminal (S470).

The payment information transmission unit 259 receives payment information from the user terminal and transmits the payment information to terminals of the plurality of veterinary pharmacies (S490). In operation S490, when the location information about the plurality of veterinary pharmacies is output to the user terminal by the pharmacy location transmission unit 257, the user inputs a veterinary medication and one or more veterinary pharmacies where the veterinary medication is available, and pays for the veterinary medication, and payment information generated in this process is received by the payment information transmission unit 259 and then transmitted to the terminals of the plurality of veterinary pharmacies.

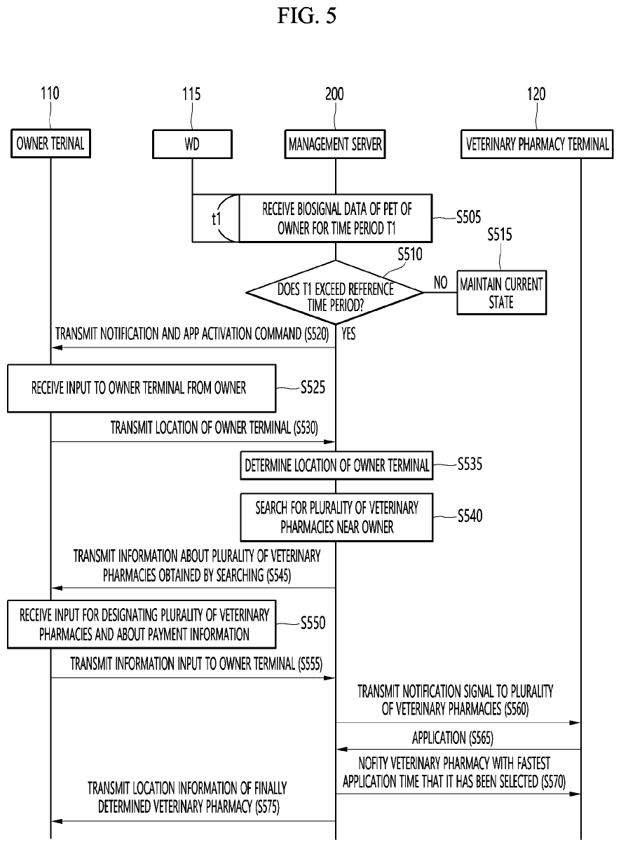

Detailed embodiments regarding the pharmacy location transmission unit 257 and the payment information transmission unit 259 will be described in detail with reference to FIG. 5.

FIG. 5 is a flowchart illustrating another example of a method according to the present disclosure.

In FIG. 5, it is assumed that the operations are performed from the top to the bottom in sequence according to time, and for convenience of description, descriptions will be provided with reference to FIGS. 1 to 3. In addition, it is assumed that the management server 200 is a server including the communication unit 230, the processing unit 250, and the like described above. In addition, in FIG. 5, the veterinary pharmacy terminal 120 is assigned the same reference numeral as the veterinary pharmacy terminal group 120 described above with reference to FIG. 1 for convenience, but is assumed to be a terminal of a veterinary pharmacy that is selected by the owner and has made an offer for the payment information with the first priority. This will be described below with reference to operations S560 to S570.

The management server 200 receives, from the WD 115, biosignal data of the pet of the owner for a time period t1 (S505). In operation S505, t1 may be the duration described above with reference to FIG. 3.

The management server 200 determines whether the duration t1 for which an abnormality in the health status of the pet is maintained exceeds a reference time period (S510), and when the duration does not exceed the reference time period, maintains the current state (S515).

When the duration t1 for which the abnormality in the health status of the pet is maintained exceeds the reference time period, the management server 200 may transmit, to the owner terminal 110, at least one of a notification and an app activation command (S520).

The owner terminal 110 receives an input thereto from the owner (S525). When the management server 200 transmits the at least one of the notification and the app activation command, a notification function in the form of a pop-up is activated in the owner terminal 110, or the owner terminal 110 operating in a standby mode (e.g., a sleep mode) transitions to a normal mode. The owner may check a notification output from the owner terminal 110 or observe that a display unit of the owner terminal 110 is controlled to be turned on, and apply a certain input to the owner terminal 110. Here, the input applied by the owner to the owner terminal 110 may be an input for executing an application related to the management server 200 or accepting a process of collecting location of the owner terminal 110.

The owner terminal 110 transmits, to the management server 200, the location information of the owner terminal 110 (S530).

The management server 200 may determine the location of the owner terminal 110 based on the location information received in operation S530 (S535), and search for a plurality of veterinary pharmacies near the owner terminal 110 (S540).

As an alternative embodiment of operation S540, the management server 200 may search for a veterinary pharmacy located within a certain distance from the owner terminal 110. According to the alternative embodiment, the owner may check location information about a veterinary pharmacy located within a walking distance from the current location of the owner, and visit the veterinary pharmacy.

As another alternative embodiment of operation S540, the management server 200 may search for a veterinary pharmacy that have a stock of the veterinary medication designated by the owner terminal 110. According to the alternative embodiment, the owner may obtain the veterinary medication as soon as he/she visits the veterinary pharmacy, and thus, the waiting time may be minimized.

As another alternative embodiment of operation S540, the management server 200 may search for a veterinary pharmacy that is out of stock of the veterinary medication designated by the owner terminal 110 but deals in the veterinary medication. The alternative embodiment may be effective for a veterinary pharmacy that is out of stock of a particular veterinary medication but intends to be selected for payment information in the order of application time and quickly stock the veterinary medication to receive a customer. For example, considering a small number of veterinary pharmacies in a rural area away from an urban area and long distances therebetween, the veterinary pharmacy may gain enough time for the customer to visit the veterinary pharmacy so as to stock the veterinary medication.

The management server 200 transmits, to the owner terminal 110, information about the plurality of veterinary pharmacies obtained by the searching (S545).

The owner terminal 110 receives an input of the owner for designating a particular veterinary pharmacy among the plurality of veterinary pharmacies received in operation S545, and pay for the veterinary medication to be purchased (S550). In operation S550, the owner performs payment for the veterinary medication corresponding to the abnormality in the health status of the pet, and in a case in which the owner tries to purchase a veterinary medication irrelevant to the detected abnormality, the management server 200 may perform control such that a message for confirming again the intention of the owner to purchase is output on the owner terminal 110.

The owner terminal 110 transmits, to the management server 200, the information input to the owner terminal 110 in operation S550 (S555).

The management server 200 transmits a notification signal to a plurality of veterinary pharmacies designated by the owner (S560). For example, when the management server 200 transmits information about ten veterinary pharmacies to the owner terminal 110, and only four of them are selected by the owner, in operation S560, the management server 200 transmits the notification signal to terminals operated by the four veterinary pharmacies.

The veterinary pharmacy terminal 120 may receive the notification signal, apply to be selected in the order of application, and become a first-priority veterinary pharmacy for the owner terminal 110 (S565).

The management server 200 transmits, to the veterinary pharmacy terminal 120 of the veterinary pharmacy that has applied in operation S565, a notification that the veterinary pharmacy has been selected to sell the veterinary medication (S570).

Thereafter, the management server 200 may transmit, to the owner terminal 110, the location information of the finally determined veterinary pharmacy (S575). Based on the location information of the veterinary pharmacy output on the owner terminal 110, the owner may visit the veterinary pharmacy and quickly obtain the veterinary medication. In this process, the management server 200 may calculate a recommended visit time considering the distance between the location of the owner terminal 110 and the location of the finally determined veterinary pharmacy, and transmit the recommended visit time to the owner terminal 110. Based on the recommended visit time, the veterinary pharmacy may stock a particular veterinary medication that is currently out of stock.

According to the present disclosure, a user may quickly obtain a veterinary medication and quickly improve the deteriorating health status of a pet.

In addition, according to the present disclosure, it is possible to prevent a case in which the user visits a veterinary pharmacy and wastes time as the medication is out of stock and thus unavailable.

In addition, the present disclosure provides a method of mediating between an owner of a pet and a veterinary pharmacy by operating a bidding system for a prepaid veterinary medication, such that the veterinary pharmacy, which is even currently out of stock of the veterinary medication but deals in the veterinary medication, is able to apply for payment information for the veterinary medication. That is, even when the veterinary pharmacy is out of stock of the veterinary medication, the veterinary pharmacy may be selected for payment information in the order of application time, quickly stock the veterinary medication, and sell the veterinary medication to a customer, thereby increasing sales.

In addition, the management server 200 of the present disclosure may primarily select a plurality of veterinary pharmacies comprehensively considering the location of the owner terminal 110, the stock of a veterinary medication selected by the owner, and whether a veterinary pharmacy that is out of stock of the veterinary medication deals in the veterinary medication, and allow the owner to secondarily select a plurality of veterinary pharmacies from among the plurality of selected veterinary pharmacies, such that the owner receives the veterinary medication that the owner wants to purchase, at the veterinary pharmacy that the owner wants.

In addition, according to the present disclosure, in the process of selecting a veterinary medication for improving the health status of the pet, the owner is able to select the veterinary medication, but when payment for a veterinary medication that is irrelevant to the health status of the pet is made, the owner may be prevented from making a purchase mistake, by outputting a message for confirming the purchase.

The embodiments of the present disclosure described above may be implemented as a computer program that may be executed through various components on a computer, and such a computer program may be recorded in a computer-readable medium. In this case, the medium may include a magnetic medium, such as a hard disk, a floppy disk, or a magnetic tape, an optical recording medium, such as a compact-disc read-only memory (CD-ROM) or a digital video disc (DVD), a magneto-optical medium, such as a floptical disk, and a hardware device specially configured to store and execute program instructions, such as ROM, random-access memory (RAM), or flash memory.

Meanwhile, the computer program may be specially designed and configured for the present disclosure or may be well-known to and usable by those skill in the art of computer software. Examples of the computer program may include not only machine code, such as code made by a compiler, but also high-level language code that is executable by a computer by using an interpreter or the like.

Particular executions described herein are merely examples and do not limit the scope of the present disclosure in any way. For the sake of brevity, conventional electronics, control systems, software and other functional aspects of the systems may not be described in detail. Furthermore, line connections or connection members between elements depicted in the drawings represent functional connections and/or physical or circuit connections by way of example, and in actual applications, they may be replaced or embodied with various suitable additional functional connections, physical connections, or circuit connections. Moreover, no item or component is essential to the practice of the present disclosure unless the item or component is specifically described as being “essential” or “critical”.

The term ‘the’ and other demonstratives similar thereto in the specification of the present disclosure (especially in the following claims) should be understood to include a singular form and plural forms. Furthermore, recitation of ranges of values herein are merely intended to serve as a shorthand method of referring individually to each separate value falling within the range, unless otherwise indicated herein, and each separate value is incorporated into the specification as if it were individually recited herein. Finally, the operations of the methods described herein may be performed in any suitable order unless otherwise indicated herein or otherwise clearly contradicted by context. The present disclosure is not limited to the described order of the operations. The use of any and all examples, or exemplary language (e.g., ‘and the like’) provided herein, is intended merely to better illuminate the present disclosure and does not pose a limitation on the scope of the present disclosure unless otherwise claimed. In addition, various modifications, combinations, and adaptations will be readily apparent to those skill in the art without departing from the following claims and equivalents thereof.

According to the present disclosure, a user may quickly obtain a veterinary medication and quickly improve the deteriorating health status of a pet.

In addition, according to the present disclosure, it is possible to prevent a case in which the user visits a veterinary pharmacy and wastes time as the medication is out of stock and thus unavailable.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A method of mediating purchase of a pet medication, the method comprising:
detecting an abnormality in health status of a pet of a user, based on information collected by a wearable device;
determining whether a duration of the detected abnormality exceeds a preset reference time period;
determining a location of a user terminal by detecting an input of the user to the user terminal after the duration of the detected abnormality exceeds the reference time period;
searching for location information of a plurality of veterinary pharmacies, based on the determined location, and transmitting, to the user terminal, a result of the searching; and
receiving payment information input to the user terminal that has received the result, and transmitting the payment information to at least one terminal of the plurality of veterinary pharmacies.

2. The method of claim 1, wherein the payment information is payment information for a veterinary medication corresponding to the abnormality.

3. The method of claim 1, wherein the determining of the location of the user terminal comprises:
when the duration exceeds the reference time period, activating a notification function of the user terminal; and
determining the location of the user terminal by detecting the input of the user to the user terminal after the notification function is activated.

4. The method of claim 1, further comprising transmitting, to the user terminal, location information of the veterinary pharmacy to which the payment information is transmitted.

5. The method of claim 1, wherein the transmitting of the payment information comprises transmitting the payment information to at least one terminal of a plurality of veterinary pharmacies that are selected in order of distance from the determined location.

6. The method of claim 1, wherein the plurality of veterinary pharmacies are in stock of the veterinary medication corresponding to the abnormality.

7. The method of claim 1, wherein the plurality of veterinary pharmacies deal in the veterinary medication corresponding to the abnormality.

8. The method of claim 1, wherein the detecting of the abnormality comprises receiving biosignals collected by the wearable device without passing through the user terminal.

9. The method of claim 1, wherein the detecting of the abnormality comprises receiving biosignals collected by the wearable device through the user terminal.

10. A computer-readable recording medium having recorded thereon a program for executing the method of claim 1.

11. A server for mediating purchase of a pet medication, the server comprising:
an abnormality detection unit configured to detect an abnormality in health status of a pet of a user, based on information collected by a wearable device;
a reference determination unit configured to determine whether a duration of the detected abnormality exceeds a preset reference time period;
a location determination unit configured to determine a location of a user terminal by detecting an input of the user to the user terminal after the duration of the detected abnormality exceeds the reference time period;
a pharmacy location transmission unit configured to search for location information of a plurality of veterinary pharmacies, based on the determined location, and transmit, to the user terminal, a result of the searching; and
a payment information transmission unit configured to receive payment information input to the user terminal that has received the result, and transmit the payment information to at least one terminal of the plurality of veterinary pharmacies.

12. The server of claim 11, wherein the payment information is payment information for a veterinary medication corresponding to the abnormality.

13. The server of claim 11, wherein the location determination unit is further configured to, when the duration exceeds the reference time period, activate a notification function of the user terminal, and determine the location of the user terminal by detecting the input of the user to the user terminal after the notification function is activated.

14. The server of claim 11, wherein the payment information transmission unit is further configured to transmit, to the user terminal, location information of the veterinary pharmacy to which the payment information is transmitted.

15. The server of claim 11, wherein the payment information transmission unit is further configured to transmit the payment information to at least one terminal of a plurality of veterinary pharmacies that are selected in order of distance from the determined location.

16. The server of claim 11, wherein the plurality of veterinary pharmacies are in stock of the veterinary medication corresponding to the abnormality.

17. The server of claim 11, wherein the plurality of veterinary pharmacies deal in the veterinary medication corresponding to the abnormality.

18. The server of claim 11, wherein the abnormality detection unit is further configured to receive biosignals collected by the wearable device without passing through the user terminal.

19. The server of claim 11, wherein the abnormality detection unit is further configured to receive biosignals collected by the wearable device through the user terminal.
